SEQUENCE LISTING (3 sequences)

SEQ ID NO: 1 - protein, 16 aa, synthetic construct
RGVPHIVMVDAYKRYK

SEQ ID NO: 2 - DNA, 20 bp, synthetic construct
  source 1..20: mol_type = other DNA
gcatcgacttcaaggaggac

SEQ ID NO: 3 - DNA, 21 bp, synthetic construct
  source 1..21: mol_type = other DNA
tgcacgctgccgtcctcgatg

(19) United States
(12) Patent Application Publication
(10) Pub. No.: US 2025/0027112 A1
(43) Pub. Date: Jan. 23, 2025

(54) GENE THERAPY DELIVERY COMPOSITIONS AND METHODS OF USE THEREOF

(71) Applicant: Washington University, St. Louis, MO (US)

(72) Inventors: Logan Collins, St. Louis, MO (US); David Curiel, St. Louis, MO (US)

(73) Assignee: Washington University, St. Louis, MO (US)

(21) Appl. No.: 18/775,975

(22) Filed: Jul. 17, 2024

Related U.S. Application Data
(60) Provisional application No. 63/513,991, filed on Jul. 17, 2023.

(51) Int. Cl. C12N 15/86; A61K 48/00; C12N 15/88

(52) U.S. Cl. CPC C12N 15/86; A61K 48/005; C12N 15/88; C12N 2750/14143; C12N 2750/14151

(57) ABSTRACT

Among the various aspects of the present disclosure is the provision of gene delivery compositions and methods of use thereof. Compositions of gene delivery matter that include adeno-associated viruses (AAVs) packaged inside vault proteins are described. Methods of treating a patient in need of a gene therapy using the disclosed AAVs packaged in vault proteins are also described.

### CROSS-REFERENCE TO RELATED APPLICATIONS

This application claims the benefit of priority to U.S. Provisional Application Ser. No. 63/513,991 filed on Jul. 17, 2023, which is incorporated by reference herein in its entirety.

### STATEMENT REGARDING FEDERALLY SPONSORED RESEARCH OR DEVELOPMENT

Not applicable.

### MATERIAL INCORPORATED-BY-REFERENCE

The Sequence Listing, which is a part of the present disclosure, includes a computer-readable form comprising nucleotide and/or amino acid sequences of the present invention (file name “020531-US-NP_sequence_listing_substitute” created on 29 Aug. 2024; 5,485 bytes). The subject matter of the Sequence Listing is incorporated herein by reference in its entirety.

### FIELD OF THE INVENTION

The present disclosure generally relates to gene delivery compositions and methods of use thereof.

### BACKGROUND OF THE INVENTION

Adeno-associated viruses (AAVs) represent one of gene therapy's success stories with three clinically approved AAV therapies on the U.S. market, dozens more coming along the development pipeline, and numerous enthusiastic efforts across academia and industry towards new ways to enhance the vector's properties. But overshadowing these successes is the fact that a large fraction of the human population (30-60%) has preexisting antibodies that neutralize most or all of the AAV serotypes in use. Many patients cannot be treated with AAVs due to a lack of efficacy and concerns about immunotoxicity. In addition, a single dose of AAV therapy generally must suffice to treat a given. disease since patients develop neutralizing antibodies after their first exposure, limiting the scope of AAV therapy even further.

Some strategies have been explored to mitigate these issues such as immunosuppression, plasmapheresis, and usage of empty decoy AAV capsids, but these approaches are complex and carry high risks to patients. To fully capitalize on the AAV's uniquely promising abilities, new ways to circumvent neutralizing antibodies are needed.

In patients without anti-AAV neutralizing antibodies, AAVs possess great potential as vaccine vectors. AAVs encoding antigens induce stronger and longer-lasting antibody responses compared with many other vaccine strategies, likely due to their stable long-term transgene expression. Further, AAV vaccines can elicit strong and specific CD8+ T cell responses. If AAVs could be applied as mucosal vaccines, such vaccines may induce safe and efficacious immunity surpassing that of other existing vectors. Mucosal vaccines in particular have immense potential in the context of COVID-19 and other respiratory infections since they can directly block disease transmission at the site of entry. Unlike intramuscular vaccination strategies, mucosal vaccines can both prevent infection in the individual and preclude the spread of the disease through populations. Unfortunately, known AAV serotypes very poorly transduce the phagocytic cells found in mucosal tissues and therefore are not thought of as well-suited for mucosal vaccine formulations.

A handful of approaches for circumventing anti-AAV neutralizing antibodies have been attempted. Capsid engineering by directed evolution and machine learning approaches have yielded antigenically distinct AAVs that evade antibodies to some level but are constrained by the locations of the capsid mutations, so some changes do not improve immune evasion and some changes reduce the infectivity of the virus. Multiple rounds of plasmapheresis coupled with pharmacological immunosuppression have shown some success, but this process is expensive, invasive, and risky. Another promising route is packaging a fraction of AAVs coming out of producer cells into exosomes (exoAAVs) thereby imparting partial protection from neutralizing antibodies. However, exoAAVs have encountered obstacles in the form of poor yields, purity issues, and difficulties in accurately determining titers for consistent therapeutic administration in animal models. Chemical modification of AAV capsids with polyethylene glycol has shown partial protection from neutralizing antibodies at the expense of substantially decreasing cellular transduction efficiency. Although some progress has been made, it thus remains clear that improved ways of overcoming neutralizing antibodies are desperately needed for AAV gene therapy to reach its full potential.

Vaults are ribonucleoprotein particles found in most eukaryotes and are thought to have been present in the last eukaryote common ancestor. The particle shell of vaults can be produced recombinantly using insect cells or yeast cells. These recombinant particles are structurally indistinguishable from native vaults except that they are hollow inside. Vaults are naturally produced in human cells and consist of 78 copies of major vault protein (MVP). Vaults are about 70 nm in length and 35 nm in diameter, making them large enough to contain an entire AAV capsid.

Recombinant vault particles have previously been repurposed as drug delivery vehicles and as vaccines. Because they are already found in human cells, they are recognized as self by the immune system and thus do not trigger immune responses. Vaults have been shown to strongly target and transduce phagocytic cells like macrophages and dendritic cells, which are found in large numbers at mucosal sites, and facilitate the efficacy of mucosal vaccines. Vaults can also be retargeted to other cell types via the attachment of antibodies or other targeting molecules to the C-terminus of MVP. Vaults carrying antigen molecules SpyCatcher003 with a third flexible linker. In some aspects, the AAV vector further includes a vector genome comprising a therapeutic gene. In some aspects, The therapeutic gene is selected from a gene therapy for a genetic disease and a gene encoding a vaccine antigen. In some aspects, the vault nanoparticle is further functionalized with a plurality of targeting molecules configured to preferentially bind to corresponding target cells.

In another aspect, a method of treating a patient in need of an AAV therapy is disclosed that includes administering a therapeutically effective amount of a composition comprising an adeno-associated virus (AAV) vector packaged inside a vault nanoparticle. The AAV vector includes a protein capsid functionalized with a plurality of INT proteins. The vault nanoparticle includes a plurality of MVP proteins, and an INT moiety of the AAV vector is covalently attached near an N-terminus of an MVP protein. In some aspects, the protein capsid includes a plurality of SpyTag proteins, a plurality of SpyCatcher proteins covalently attached to the plurality of Spytag proteins, and a plurality of INT proteins covalently attached to the plurality of SpyCatcher proteins. In some aspects, the SpyTag protein includes SpyTag003 protein and the SpyCatcher protein includes SpyCatcher003 protein. In some aspects, the SpyTag003 protein is flanked by a first and second flexible linker comprising the amino acid sequences AGGGSGGS (SEQ_ID_NO: 4) and AGGGSGGS (SEQ_ID_NO: 4), respectively. In some aspects, the INT peptide is covalently attached to the SpyCatcher003 with a third flexible linker. In some aspects, the AAV vector further includes a vector genome that includes a therapeutic gene. In some aspects, the therapeutic gene is selected from a gene therapy for a genetic disease and a gene encoding a vaccine antigen. In some aspects, the vault nanoparticle is configured to prevent the AAV vector from a neutralizing antibody produced by an immune response to the AAV therapy. In some aspects, the vault nanoparticle is further functionalized with a plurality of targeting molecules configured to preferentially bind to corresponding target cells.

In an additional aspect, a method for producing an AAV therapy composition comprising an adeno-associated virus (AAV) vector packaged inside a vault nanoparticle is disclosed. The method includes providing a plurality of AAV vectors, a plurality of fusion proteins, and a plurality of vault nanoparticles comprising a plurality of MVT proteins. Each AAV vector includes a protein capsid functionalized with a plurality of SpyTag proteins flanked by first and second flexible linkers. Each fusion protein includes an INT protein covalently linked to a SpyCatcher protein by a third flexible linker. The method further includes mixing the plurality of AAV vectors with the plurality of fusion proteins to form a plurality of INT-functionalized AAV vectors, in which each INT-functionalized AAV vector includes an AAV vector and the SpyCatcher proteins of at least a portion of the fusion proteins covalently attached to the SpyTag proteins of the protein capsid. The method further includes incubating the INT-functionalized AAV vectors with the plurality of vault nanoparticles to form the AAV therapy composition, in which at least a portion of the INT proteins of each INT-functionalized AAV vector is covalently attached to at least a portion of the MVP proteins of each vault nanoparticle. In some aspects, the plurality of AAV vectors is mixed with the plurality of fusion proteins at a copy ratio of 1 copy of an AAV vector to 10 copies of the fusion protein. In some aspects, the INT-functionalized AAV vectors are incubated with the vault nanoparticles at a copy ratio of 1 copy of the INT-functionalized AAV vector to 5 copies of the vault nanoparticles. In some aspects, the SpyTag protein includes SpyTag003 protein, the SpyCatcher protein includes SpyCatcher003 protein, and the first and second flexible linkers include the amino acid sequences AGGGSGGS (SEQ_ID_NO: 4) and AGGGSGGS (SEQ_ID_NO: 4), respectively.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A is a schematic diagram illustrating the structure of the outer shell of a protein vault complex (PDB 4V60); the vault shell consists of 78 copies of major vault protein (MVP) assembled into a hollow compartment.

FIG. 1B is a schematic diagram illustrating a process of packaging AAV into recombinant vaults by producing an AAV9 with SpyTag003 inserted into its VP2 capsid protein (AAV-SpT), creating a SpyCatcher003-INT fusion protein (SpC-INT), conjugating the SpC3-INT with the AAv9-VP2-SpT3, and mixing the AAV-INT particles with empty vaults and incubating overnight to induce packaging.

FIG. 1C is a set of transmission electron microscopy (TEM) images of vault-AAV complexes.

FIG. 1D is a schematic diagram illustrating vault AAV complexes transducing a cell despite the presence of anti-AAV neutralizing antibodies. The vault appears to behave as an immunologically invisible shield. After uptake, the vault disassembles within the acidifying endosome, the AAV exposes its VP1 phospholipase A2 domain to facilitate endosomal escape, and the AAV experiences transport to the nucleus.

FIG. 2A is a schematic diagram illustrating an AAV9-VP2-SpT3 design which includes SpT3 flanked by flexible linkers and inserted into the VP2 capsid protein. To prevent the SpT3 from appearing in VP1 and VP3, two separate plasmids were used to encode (i) VP2-SpT3 and to encode (ii) VP1 and VP3 during AAV production. SpC3-INT was designed as a C-terminal fusion of INT peptide onto SpC3, bridged by a flexible linker.

FIG. 2B is an image of an SDS-PAGE validating the AAV9-SpT construct by comparing AAV9-SpT alone to AAV9-SpT that was reacted with SPC-Cas9.

FIG. 2C is a schematic of genetically inserting SpyTag003 (SpT3) flanked by flexible linkers into the VP2 protein of AAV9.

FIG. 2D is an image of a Western blot of the AAV capsid proteins that validated the successful production of wild-type AAV9, AAV9-VP2-SpT1, and AAV9-VP2-SpT3. The VP2-SpT1 and VP2-SpT3 proteins show the expected 1 kDa shift on the gel relative to VP2. AAV9-VP2-SpT1 was included as an additional control.

FIG. 2E is an image of SDS-PAGE validating the SpC3-INT protein.

FIG. 3A is a set of TEM images of AAV9-SpC-INT mixed with vaults at a 1:1 ratio and incubating overnight at room temperature. The AAV capsids spontaneously package into vaults with high efficiency (left). By comparison, AAV9 without the INT peptide (right) does not package into vaults.

FIG. 3B is a set of representative TEM images of vault-AAV complexes.

FIG. 4A is a schematic illustrating an experimental workflow for intranasal vaccination and analysis.

FIG. 4B is a pair of graphs demonstrating the increase in serum antibodies S protein (left) and RBD (right) after intranasal vaccination via ELISA.

FIG. 5A is a set of TEM images of purified AAV (top) and purified vault (bottom) samples.

FIG. 5B is a set of representative TEM images of vault/AAV complexes (VAAV).

FIG. 5C is a set of TEM images comparing a VAAV sample (left) and a control vault+AAV sample (right) wherein the latter contains a mixture of vaults with AAV9-VP2-SpT3 conjugated to SpC3. In the VAAV sample, a substantial fraction of AAV-INT undergoes packaging by vaults. In the vault+AAV sample, the AAVs lack the INT peptide and thus do not experience packaging into vaults.

FIG. 5D is a representative TEM image showing that although VAAV packaging occurs with reasonably high frequency, many unpackaged AAVs remain present in the mixture. Packaged VAAVs are marked with arrows.

FIG. 6A is a set of images of tomogram slices of VAAV and vault particles, showing the three views in orthogonal directions (Y, Z, and X). Arrows show vaults and AAVs.

FIG. 6B is a 3D rendering of a tomogram with a VAAV particle with a wedge element removed to reveal the AAV contained within the vault.

FIG. 7A is a set of representative fluorescence microscopy images showing VAAV cellular transduction in the presence (top row) and absence (bottom row) of neutralizing serum. VAAV (left column) transduces cells in the presence of neutralizing serum, vault+AAV (left center column) transduces cells to a low level in the presence of neutralizing serum, AAV alone (right center column) does not transduce cells in the presence of neutralizing serum, and the PBS group (right column) shows no fluorescence. Both VAAV and vault+AAV very strongly transduce cells in the absence of neutralizing serum, AAV alone transduces cells to a moderate extent in the absence of neutralizing serum, and the PBS group shows no fluorescence.

FIG. 7B is a graph of flow cytometric quantification of VAAV, vault+AAV, AAV, and PBS group transduction of cells in the presence of neutralizing serum as quantified from images similar to FIG. 7A.

FIG. 7C is a graph of flow cytometric quantification of VAAV, vault+AAV, AAV, and PBS group transduction of cells in the absence of neutralizing serum as quantified from images similar to FIG. 7A.

### DETAILED DESCRIPTION OF THE INVENTION

The present disclosure is based, at least in part, on the discovery that entire virus capsids including, but not limited to adeno-associated viruses (AAVs) can be packaged inside of protein vaults for gene delivery applications. As shown herein, gene delivery systems that make use of viruses contained within protein compartments are described.

One aspect of the present disclosure provides for a gene delivery vehicle that can include a virus, which can be but is not limited to adeno-associated and protein compartments, which can be but are not limited to vault proteins.

In brief, the present disclosure is directed to a composition of matter that involves the delivery of an adeno-associated virus (AAV) to the interior of vault structures. Vault is a naturally occurring hollow protein structure consisting of copies of the major vault protein (MVP), where the AAV can be covalently linked to INT, a peptide with high specificity to the interior sites of vault. In the present disclosure, the AAV acts as cargo to be delivered by vault to varying cell types, particularly that of the mucosal epithelium tissue. This AAV-Vault combination is able to circumvent significant anti-AAV targeting in patients and additionally enable the targeting of mucosal tissue which AAVs natively lack and thus have potential applications as a platform for vaccine delivery.

In some aspects, the present disclosure describes adeno-associated viruses (AAVs) packaged inside protein vault nanocompartments as a new type of delivery system for gene therapy. In another aspect, these vault-AAV complexes can evade preexisting immunity, overcoming a key challenge in AAV-mediated gene therapy. In one aspect, transmission electron microscopy has demonstrated that AAVs can be packaged inside vaults.

Protein vaults are synthesized by human cells, so they are considered “self” by the immune system. For this reason, protein vaults can act as protective shells that shield AAVs from preexisting immunity. Vaults can be taken up into human cells by endocytosis and disassembled in the endosome. In one aspect, this cellular uptake allows the AAVs to escape the endosome (using their VP2 phospholipase) and deliver their DNA into the nucleus.

Adeno-associated virus (AAV) gene therapy is plagued by issues of preexisting immunity in a large fraction of patients leading to toxicity and low therapeutic efficacy. By encapsulating AAVs, vaults allow the viruses to evade the immune system. Vaults are also targetable for uptake by a variety of cell types, though they have particularly good natural tropism for dendritic cells and macrophages. Vault-AAV complexes can thus facilitate safer and more effective AAV gene therapies that are highly applicable to cancer immunotherapy and treating immune disorders, yet also have great promise for combating a wide range of other types of illnesses.

In some embodiments, fusion protein SpyCatcher003-INT can be employed in combination with AAV9-VP2-SpyTag003 to make AAV9SpT-SpCINT, which packages more efficiently than AAV9 on its own.

In various aspects, the gene delivery system may include any suitable human or non-human AAV serotype without limitation. Non-limiting examples of suitable AAV serotypes for use in the gene delivery system include AAV1, AAV2, AAV3, AAV4, AAV5, AAV6, AAV7, AAV8, AAV9, AAV10, AAV11, AAV-DJ, AAV-Rh10, AAV-retro, AAV.PHP.B, AAV.PHP.eB, AAV.PHP.S, AAV.CAP-B10, and AAV.CAP-B22. In another aspect, any AAV serotype with a self-complementary genome (scAAV) can be used. In yet another aspect, any naturally occurring or engineered AAV serotype can be used.

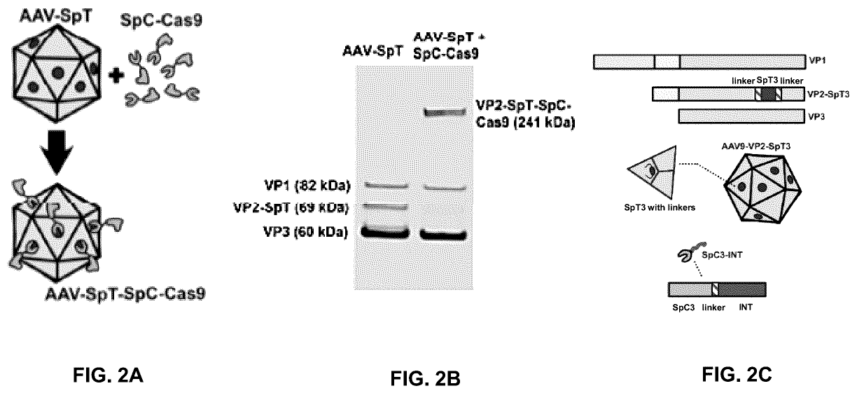

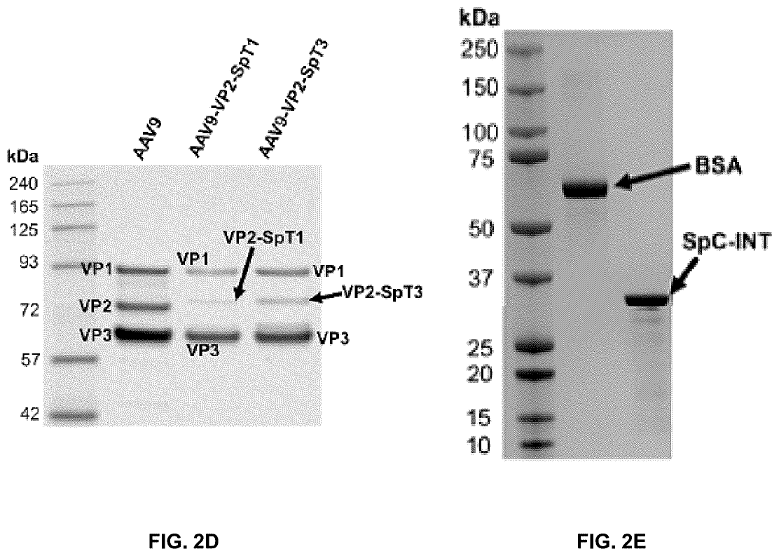

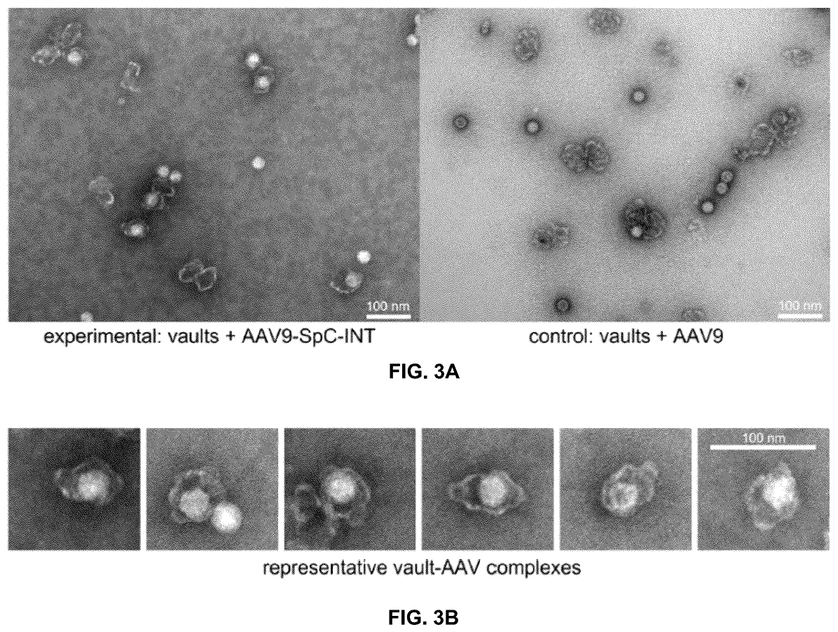

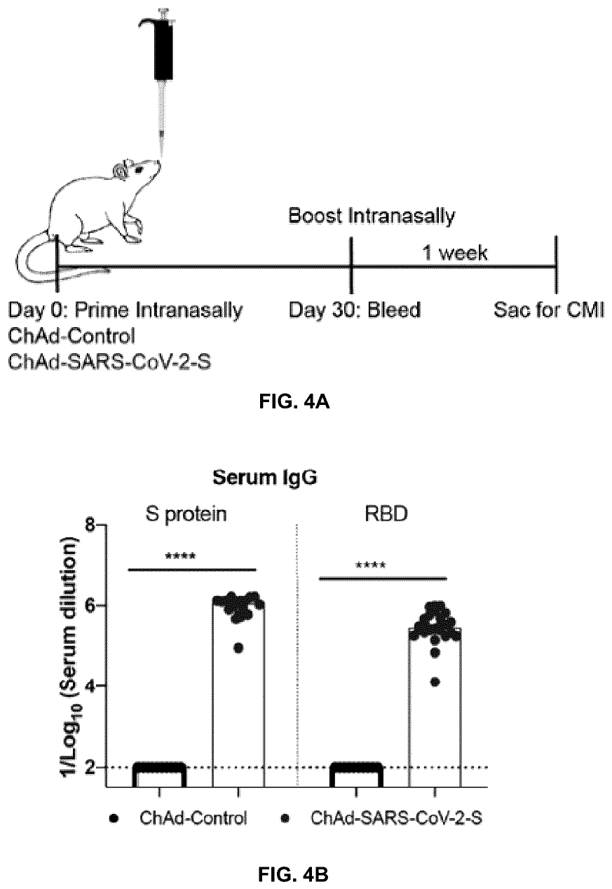

The feasibility of packaging AAV inside of protein vaults by covalently linking INT peptide onto AAV capsids and then mixing the AAV-INT capsids with vaults at a 1:1 ratio and incubating at room temperature overnight has been demonstrated. To conjugate INT onto AAV, the SpyTag-SpyCatcher system was utilized. This pair of small proteins rapidly and specifically reacts to form a covalent isopeptide bond. AAV9 bearing a SpyTag003 peptide inserted into its VP2 protein (hereafter referred to as AAV9-SpT) was employed. This AAV9-SpT was validated by reacting with a SpC-Cas9 fusion protein reagent that had been employed in a past study and then utilizing SDS-PAGE (FIG. 2A-B in Appendix A). A SpyCatcher003-INT fusion protein (SpC-INT) was then synthesized. The SpC-INT was also validated by SDS-PAGE (FIG. 2C in Appendix A). By mixing enough SpC-INT with the AAV9-SpT to react with three SpT sites per AAV capsid, AAV9-SpC-INT (hereafter referred to as AAV9-INT) was created. Next, the AAV9-INT was mixed with vaults at a 1:1 capsid-to-vault particle ratio and incubated at room temperature overnight. Packaging of AAVs into vaults was validated using negative stain transmission electron microscopy (TEM) and compared to a control in which AAV9 without INT was mixed with vaults (FIG. 3A-B in Appendix A). In the experimental group, a substantial fraction of vaults had spontaneously packaged an AAV9-INT capsid, whereas in the control group, AAV9 was not observed inside the vaults. Similar results were observed upon repetition of the experiment. This demonstrates the technical feasibility of packaging entire virus capsids inside protein vaults.

Although the INT functionalization of the AAV capsid is disclosed herein in terms of SpyTag-SpyCatcher chemistry, this functionalization may be accomplished using any suitable covalent binding chemistry or system without limitation. In various aspects, the AAV capsid may be covalently bound to the C-terminus of the MVP proteins of the vault nanoparticle using SpyTag-SpyCatcher chemistry, streptavidin-biotin chemistry, DogTag-DogCatcher chemistry, SnoopTag-SnoopCatcher chemistry and any other any suitable binding chemistry without limitation. By way of non-limiting example, the AAV capsid may be selectively biotinylated and incubated with an INT-streptavidin fusion protein. By way of other non-limiting examples, the AAV capsid may be inserted with any one of a SpyTag, DogTag, or SnoopTag sequence and cultured with a fusion protein that includes INT and the corresponding SpyCatcher, DogCatcher, or SnoopCatcher proteins.

In various aspects, the disclosed gene delivery system may be targeted to specific cells or tissues by functionalizing the vault nanoparticle with a plurality of targeting molecules using a binding chemistry similar to the binding chemistries used to attach the AAV capsid to the vault particle as described above. Non-limiting examples of suitable binding chemistries include SpyTag-SpyCatcher chemistry, streptavidin-biotin chemistry, DogTag-DogCatcher chemistry, SnoopTag-SnoopCatcher chemistry, and any other suitable binding chemistry without limitation. Non-limiting examples of suitable targeting molecules include an antibody, an scFv, a nanobody, a peptide ligand, an aptamer, and any other suitable targeting molecule without limitation. By way of non-limiting example, the C-terminus of the MVP proteins of the vault nanoparticle may be attached or linked to a binding element including but not limited to, SpyTag, DogTag, or SnoopTag and cultured with a fusion protein that includes the targeting molecule fused or linked to the corresponding SpyCatcher, DogCatcher, or SnoopCatcher protein.

In some aspects, the disclosed gene delivery system may be used as a vaccine, wherein the vector genome of the AAV is modified to express a vaccine antigen. In other aspects, the disclosed gene delivery system may be used to enable various gene therapies including, but not limited to, alpha-1 antitrypsin (A1AT) deficiency replacement therapy wherein the vector genome encodes the A1AT protein and delivers to lungs, CAR T cell therapy wherein the vector genome encodes the CAR protein and delivers to T cells, spinal muscular atrophy AAV gene therapy, hemophilia A or B AAV gene therapy, muscular dystrophy AAV gene therapy, and Leber's congenital amaurosis AAV gene therapy.

### Gene Modulation Agents

As described herein, gene expression has been implicated in various diseases, disorders, and conditions. As such, modulation of genes (e.g., modulation of genes through a gene delivery system) can be used for the treatment of such conditions. A gene modulation agent can modulate gene response or induce or inhibit gene expression. Gene modulation can comprise modulating the expression of genes on cells, modulating the quantity of cells that express genes, or modulating the quality of the gene-expressing cells.

Gene modulation agents can be any composition or method that can modulate gene expression on cells (e.g., by delivering a gene delivery agent). For example, a gene modulation agent can be an activator, an inhibitor, an agonist, or an antagonist. As another example, the gene modulation can be the result of gene editing.

A gene modulation agent can be an antibody (e.g., a monoclonal antibody to a gene product).

A gene modulating agent can be an agent that induces or inhibits progenitor cell differentiation into gene expressing cells.

Gene Signal Reduction, Elimination, or Inhibition by Small Molecule Inhibitors, shRNA, siRNA, or ASOs

As described herein, a gene modulation agent can be used for use in a therapy for a disease. A gene modulation agent can be used to reduce/eliminate or enhance/increase gene signals. For example, a gene modulation agent can be a small molecule inhibitor of a particular gene. As another example, a gene modulation agent can be a short hairpin RNA (shRNA). As another example, a gene modulation agent can be a short interfering RNA (siRNA).

As another example, RNA (e.g., long noncoding RNA (lncRNA)) can be targeted with antisense oligonucleotides (ASOs) as a therapeutic. Processes for making ASOs targeted to RNAs are well known; see e.g. Zhou et al. 2016 Methods Mol Biol. 1402:199-213. Except as otherwise noted herein, therefore, the process of the present disclosure can be carried out in accordance with such processes.

### Gene Inhibiting Agent

One aspect of the present disclosure provides for the targeting of a gene, its receptor, or its downstream signaling. The present disclosure provides methods of treating or preventing a disease based on the discovery that AAVs can be packaged into protein vaults to enhance the delivery of genes to a patient with a disease.

As described herein, inhibitors of a gene (e.g., antibodies, fusion proteins, small molecules) can reduce or prevent disease. A gene inhibiting agent can be any agent that can inhibit a gene, downregulate a gene, or knockdown a gene.

As an example, a gene inhibiting agent can inhibit gene signaling.

For example, the gene inhibiting agent can be an antibody toward a gene product associated with a disease. Furthermore, the antibody can be a murine antibody, a humanized murine antibody, or a human antibody.

As another example, the gene inhibiting agent can be an antibody, wherein the antibody prevents binding of a gene product to its receptor or prevents activation of a gene or its downstream signaling.

As another example, the gene inhibiting agent can be a fusion protein. For example, the fusion protein can be a decoy receptor for a gene product. Furthermore, the fusion protein can comprise a mouse or human Fc antibody domain fused to the ectodomain of a decoy receptor.

As another example, a gene inhibiting agent can be an inhibitory protein that antagonizes a gene or its product. For example, the gene inhibiting agent can be a viral protein, which has been shown to antagonize a gene.

As another example, a gene inhibiting agent can be a short hairpin RNA (shRNA) or a short interfering RNA (siRNA) targeting a gene or its downstream signals.

As another example, a gene inhibiting agent can be a sgRNA targeting a gene or its downstream signaling.

Methods for preparing a gene inhibiting agent (e.g., an agent capable of inhibiting gene signaling) can comprise the construction of a protein/Ab scaffold containing the natural gene receptor as a neutralizing agent; developing inhibitors of the gene receptor “down-stream”; or developing inhibitors of the gene production “up-stream”.

Inhibiting a gene can be performed by genetically modifying at least one gene in a subject or genetically modifying a subject to reduce or prevent expression of the gene, such as through the use of CRISPR-Cas9 or analogous technologies, wherein, such modification reduces or prevents a disease.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs the transcription of a nucleic acid. An inducible promoter is generally understood as a promoter that mediates the transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as many as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site, all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects the expression of the coding DNA sequence (i.e., that the coding sequence or functional RNA is under the transcriptional control of the promoter). Coding sequences can be operably linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells and organisms comprising transgenic cells are referred to as “transgenic organisms”.

“Transformed,” “transgenic,” and “recombinant” refer to a host cell or organism such as a bacterium, cyanobacterium, animal, or plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid that is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. The amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tm) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tm=81.5° C.+16.6 (log 10 [Na+])+0.41 (fraction G/C content)−0.63 (% formamide)−(600/l). Furthermore, the Tm of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated into the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged (Acidic): | D E |

Conservative Substitutions III

| Original Residue | Exemplary Substitution |
|---|---|
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or DNA generated externally, such as a DNA sequence containing an antisense message of a gene, or a DNA sequence encoding a synthetic or modified version of a gene.

Host strains developed according to the approaches described herein can be evaluated by any number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Genome Editing

As described herein, gene signals can be modulated (e.g., reduced, eliminated, or enhanced) using genome editing. Processes for genome editing are well known; see e.g. Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of a gene by genome editing can result in protection from autoimmune or inflammatory diseases.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N) 20NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be a useful tool for therapeutic applications for a disease to target cells by the removal of gene signals.

For example, the methods as described herein can comprise a method for altering a target polynucleotide sequence in a cell comprising contacting the polynucleotide sequence with a clustered regularly interspaced short palindromic repeats-associated (Cas) protein.

### Formulation

The agents and compositions described herein can be formulated in any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic agents, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce the dosage frequency. Controlled-release preparations can also be used to affect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of the agent being metabolized or excreted from the body. The controlled release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the treatment of the disease, disorder, or condition.

### Therapeutic Methods

Also provided is a process of treating, preventing, or reversing a disease in a subject in need of administration of a therapeutically effective amount of a gene modulation agent, so as to treat a disease.

Methods described herein are generally performed on a subject in need thereof. A subject in need of the therapeutic methods described herein can be a subject having, diagnosed with, suspected of having, or at risk for developing a disease. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a gene modulation agent is, for example, an amount that would cause the desired therapeutic effect in a subject while minimizing undesired side effects. In various embodiments, an effective amount of a gene modulation agent described herein can substantially inhibit a disease, slow the progress of a disease, or limit the development of a disease.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a gene modulation agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to treat a disease.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of any number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a gene modulation agent can occur as a single event or over a time course of treatment. For example, a gene modulation agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accordance with the methods described herein can be performed prior to, concurrent with, or after conventional treatment modalities for a disease.

A gene modulation agent can be administered simultaneously or sequentially with another agent, such as an antibiotic, an anti-inflammatory, or another agent. For example, a gene modulation agent can be administered simultaneously with another agent, such as an antibiotic or an anti-inflammatory. Simultaneous administration can occur through the administration of separate compositions, each containing one or more of a gene modulation agent, an antibiotic, an anti-inflammatory, or another agent. Simultaneous administration can occur through the administration of one composition containing two or more of a gene modulation agent, an antibiotic, an anti-inflammatory, or another agent. A gene modulation agent can be administered sequentially with an antibiotic, an anti-inflammatory, or another agent. For example, a gene modulation agent can be administered before or after the administration of an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well-known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10:0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve the taste of the product; or improve the shelf life of the product.

### Screening

Also provided are methods for screening.

The subject methods find use in the screening of a variety of different candidate molecules (e.g., potentially therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about-2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of compounds during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

### Kits

Also provided are kits. Such kits can include an agent or composition described herein and, in certain embodiments, instructions for administration. Such kits can facilitate the performance of the methods described herein. When supplied as a kit, the different components of the composition can be packaged in separate containers and admixed immediately before use. Components include, but are not limited to an adeno-associated virus, vault proteins, and solubilizers. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing the activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet website specified by the manufacturer or distributor of the kit.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—AAV-Vault Gene Delivery System

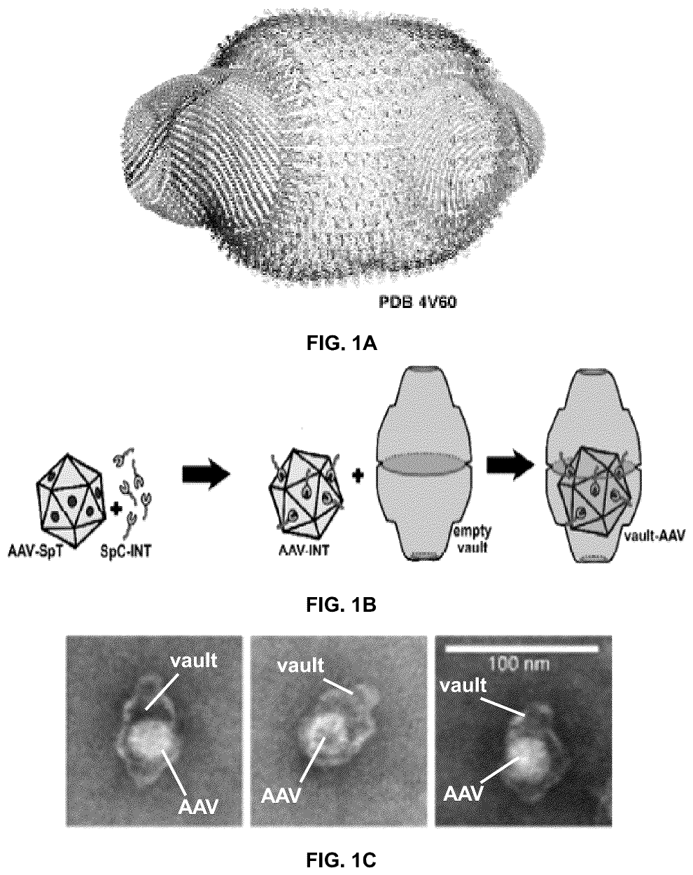

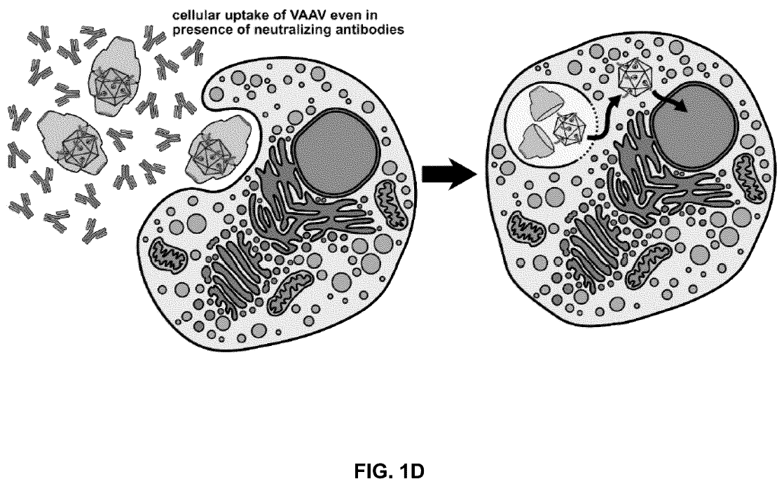

To develop a delivery system for AAVs that circumvents neutralizing antibodies from serum and conveys enhanced cellular transduction efficiency, the following experiments were conducted. SpyTag-SpyCatcher technology was used to decorate AAV with INT peptide to facilitate its packaging inside recombinant vaults, resulting in vault-AAV (VAAV) consisting of an AAV packaged inside a vault protein shell, as illustrated in FIG. 1A, FIG. 1B, FIG. 1C, and FIG. 1D). As demonstrated below, VAAV transduces cultured cells even in the presence of neutralizing serum. VAAV technology represents a strategy for overcoming the neutralizing antibody problem of AAV gene therapy.

### Methods

### Acquisition of Anti-AAV9-VP2-SpT3 Serum and SpC3-INT

Intramuscular injection of 5×1011 vg of AAV9-VP2-SpT3 was performed on C57BL/6 mice to induce the production of anti-AAV-VP2-SpT3 serum. Serum was recovered and used in subsequent experiments. Neutralization was validated by incubating varying dilutions of serum with 106 vg of AAV9-VP2-SpT3 for 1 hour at room temperature before transferring each of the samples onto 104 CHO-Lec2 cells in a 96-well plate (106 multiplicity of infection or MOI). At 4 days post-infection, fluorescence microscopy was utilized to observe what concentration of serum was necessary for full neutralization. As a custom service, GenScript produced SpC3-INT and validated it using SDS-PAGE.

### Production of AAV9-VP2-SpT3

AAV Rep-Cap plasmids containing the Rep gene from AAV2 and the Cap gene for AAV9 were produced by gene synthesis and molecular cloning. Plasmid pVP13 (plasmid identification number 1886) contains a mutation in the ACG start codon of VP2 to GCG so as to only enable expression of VP1 and VP3. Plasmid pVP2-SpT3 (plasmid identification number 3007) contains ATG to CTG mutations at the start codons of both VP1 and VP3 so as to express only the SpyTag003-modified VP2. The following amino acids were inserted after glycine 453 in the Cap sequence: flexible linker [AGGGSGGS] (SEQ_ID_NO: 4), SpyTag003 [RGVPHIVMVDAYKRYK] (SEQ_ID_NO: 1), and flexible linker [GGSGGSA] (SEQ_ID_NO: 5). Transgene plasmid pAAV-CB-EGFP (plasmid identification number 1161) (PMID: 34141821) expresses enhanced green fluorescent protein driven by the Chicken Beta Actin promoter with a CMV enhancer element. A plasmid expressing the Adenoviral helper genes required for AAV packaging (pAdDeltaF6) was obtained from the University of Pennsylvania Vector Core. AAV9-VP2-SpT3 was produced through quadruple transfection of 293T cells (ATCC, CRL-3216) using polyethylenimine (PEI) (PMID: 23791963). Transgene, helper, and Rep-Cap plasmids were supplied in an equimolar (1:1:1:1) ratio. At 72 hours post-transfection, cell pellets were harvested and recombinant AAV was purified by iodixanol gradient ultracentrifugation. Fractions containing AAV genomes were identified by qPCR, pooled, and dialyzed against PBS using a 100 kDa Spectra-Por Float-A-Lyzer G2 dialysis device (Spectrum Labs, G235059). Purified AAV was concentrated using a Sartorius Vivaspin Turbo 4 Ultrafiltration Unit (VS04T42) and stored at −80 C. AAV titers were calculated by qPCR relative relative to a standard curve of transgene plasmid, after DNase digestion to remove unencapsidated DNA [PMID: 23912992]. The following primers were used to detect the EGFP transgene for titer: for 5′ GCATCGACTTCAAGGAGGAC-3′ (SEQ_ID_NO: 2), rev 5′-TGCACGCTGCCGTCCTCGATG-3′ (SEQ_ID_NO: 3).

### Production of Vault

Codon-optimized DNA encoding human MVP was inserted into vector pJGG (BioGrammatics, San Diego, CA) and transformed into yeast (Pichia pastoris) cells. MVP-expressing P. pastoris was cultured in YPD medium at 30° C. in shake flasks at 200 rpm for 24 hours. Harvested cells were resuspended in Buffer (50 mM Tris pH 7.4, 75 mM NaCl, 0.5 mM MgCl2) supplemented with 1 mM DTT and protease inhibitor cocktail (Sigma-Aldrich P8215) and lysed by agitating with 0.5 mm glass beads. Cellular debris and beads were removed by centrifugation at 3000 g for 5 minutes followed by centrifugation at 20,000 g for 20 minutes. The supernatant was further centrifuged at 37,000 rpm for 1 hour, and the vault-containing pellet was resuspended in Buffer containing 1% Triton X-100. Vaults were precipitated by the addition of 0.1 g ammonium acetate per mL and agitated overnight at 4° C. The precipitate was collected by centrifugation at 20,000 g for 20 minutes, resuspended in Buffer, and the remaining debris was removed by centrifugation at 3000 g for 10 minutes. The supernatant was applied to a TMAE Fractogel HiCap column (EMD) and eluted via NaCl gradient. Vault-containing fractions were pelleted at 37,000 rpm for 1 hour and resuspended to a concentration of 1 mg/mL in phosphate-buffered saline containing 10 mg/mL trehalose. Aliquots of resuspended vaults were frozen in liquid nitrogen, lyophilized, and stored at −20° C.

Packaging of AAV into Vault

For the VAAV group, AAV9-VP2-SpT3 was mixed with SpC3-INT at a 1:10 copy number ratio so that the average of 5 SpT3 sites per capsid would be saturated while not having such an excess as to crowd the interiors of the vaults in the next step. For the vault+AAV control, AAV9-VP2-SpT3 was mixed with the same amount of SpC3. For the AAV control, another aliquot of AAV9-VP2-SpT3 was mixed with the same amount of SpC3-INT. The mixtures were incubated for 1 hour at 25° C. For the VAAV group and the vault+AAV control, a 5-fold copy number excess of vault relative to AAV particles was then added. The number of vaults per volume was calculated based on the molecular weight of a single vault and total vault mass resuspended in PBS with 10% glycerol. For the AAV control, an equivalent volume of PBS with 10% glycerol was added. All three mixtures were incubated at 25° C. overnight. To validate packaging, a small amount of each group was removed, diluted 1:30 in PBS with 10% glycerol, and imaged via negative stain (1% uranyl acetate) TEM. The exact number of AAV particles removed from each sample for this validation was calculated to ensure accurate MOI calculations for the subsequent experiment.

### CryoET and Tomogram Reconstruction

To begin the cryoET process, 10 nm fiducial gold beads were added to the VAAV-containing sample. An aliquot of 3.5 μL of the mixture was briefly applied onto Quantifoil holey carbon grids (300 mesh, 1.2/1.3) with a 2 nm continuous carbon layer that was glow-discharged for 60 s at −25 mA with PELCO easiGlow. After a 2-minute waiting time, the extra sample on the grids was blotted away by filter paper and the resulting cryoEM grid was placed in an FEI/Thermo-Fisher Mark IV Vitrobot cryo-sample plunger at blot force 2 and blot time 3 s. The grids were next plunged into a precooled liquid ethane/propane mixture for vitrification. Plunge-freezing conditions and the concentrations of vaults and AAVs were optimized by screening with an FEI TF20 TEM equipped with a Gatan K3 camera.

Tilt series were collected using SerialEM in a FEI Titan Krios transmission electron microscope equipped with an energy filter and a Gatan K3 camera at super-resolution mode. The collecting magnification was 64 kx and the tilt range was −60° to 60° with a 3° tilt-series increment and dose symmetry. Total electron exposure for one tilt series was 120 e-/Å2 with frame fraction. Frames in the tilt series were drift-corrected using MotionCor2 and the corrected micrographs were aligned and reconstructed into tomograms with AreTomo. IsoNet was applied to enhance the contrast and partially alleviate the missing wedge issue. Visualization of tomogram slices and three-dimensional rendering of tomograms was performed using IMOD and UCSF ChimeraX.

Infection Assay with and without Neutralizing Serum

Groups including VAAV, vault+AAV, AAV, and PBS were prepared and validated by TEM prior to the infection experiment. CHO-Lec2 cells were seeded at 104 cells per well in a 96-well plate. For the groups with neutralizing serum, a 1:100 dilution of serum was mixed with VAAV, vault+AAV, AAV, and PBS samples 1 hour before transferring the mixtures to appropriate wells in triplicate at an MOI of 106 vg per cell. (Samples were incubated at room temperature during this 1-hour period). The final dilution of serum in the wells was 1:200. For the groups without neutralizing serum, mixtures of VAAV, vault+AAV, AAV only, and PBS were prepared and added to the appropriate wells in triplicate at an MOI of 106 vg per cell. At 4 days post-infection, the cells were imaged by fluorescence microscopy. Flow cytometry was then employed to quantify the percentage of eGFP-positive cells in each well. A one-way ANOVA (analysis of variance) test was used to determine statistical significance.

### Results

As a way of site-specifically conjugating INT onto AAV, the molecular glue peptides SpyTag-SpyCatcher were used to covalently link INT onto AAV prior to mixing the AAV-INT with vault particles (FIG. 2A) and validated by SDS-PAGE (FIG. 2B). The SpyTag003 (SpT3) was genetically inserted and flanked by flexible linkers into the VP2 protein of AAV9 (FIG. 2C) and confirmed incorporation of VP2-SpT3 into the capsid by Western blot (FIG. 2D). Because each AAV capsid on average contained five copies of VP2 out of a total of sixty capsid proteins, we reasoned that this would minimally disrupt viral production and function. We next designed a SpyCatcher003-INT (SpC3-INT) fusion protein wherein a flexible linker connected the SpC3 and the INT peptide (FIG. 2C). We validated the SpC3-INT protein by SDS-PAGE (FIG. 2E). After this, we mixed AAV9-VP2-SpT3 with SpC3-INT to induce the spontaneous formation of AAV9-VP2-SpT3-SpC3-INT, hereafter referred to as AAV9-INT.

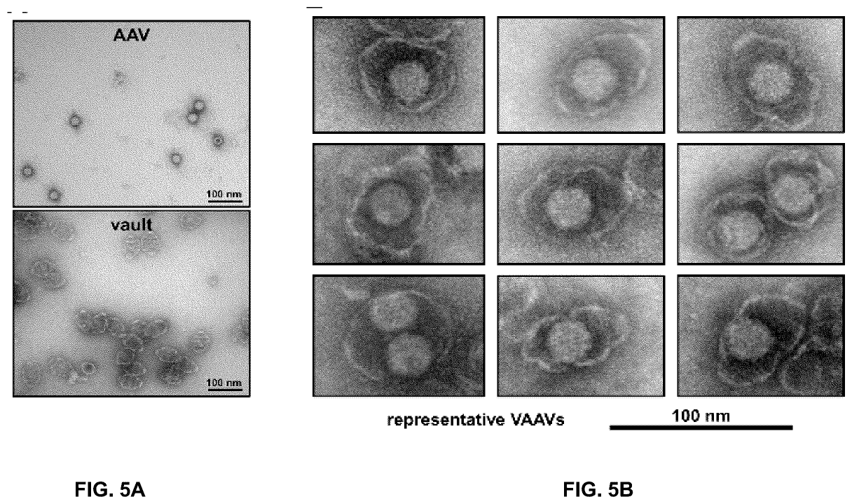

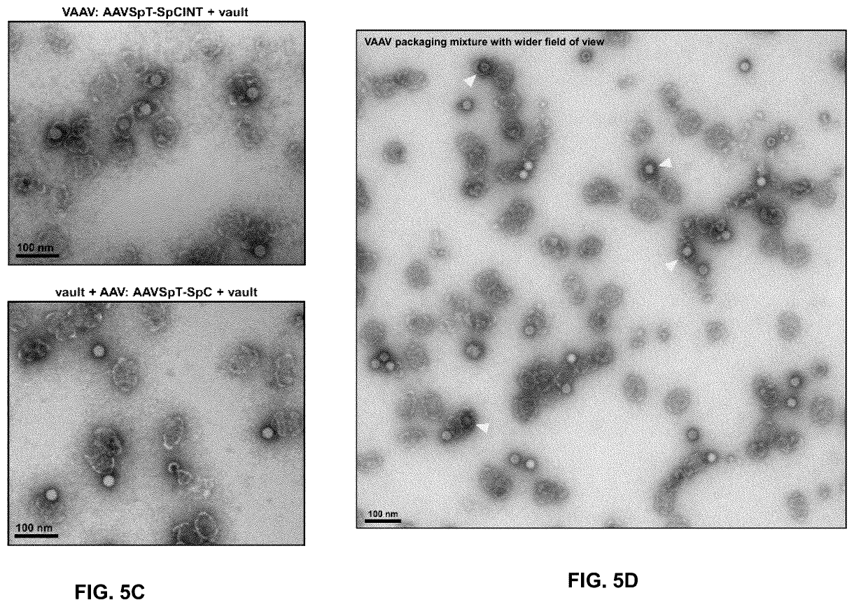

The AAV9-INT was mixed with purified vaults at a 1:5 particle count ratio and incubated overnight at room temperature to facilitate packaging. Since vaults spontaneously package INT-bearing biomolecules, it was anticipated they might be able to encapsulate AAV-INT despite its larger size and complexity compared to past vault cargos. The AAV and vaults were imaged alone to verify their morphological characteristics (FIG. 5A). After mixing AAV-INT with vaults, it was determined using transmission electron microscopy (TEM) imaging overnight incubation at room temperature was sufficient to induce packaging of a sizable fraction of AAVs into vaults, though many free AAVs did remain unencapsulated (FIG. 3A, FIG. 3B, FIG. 5D). Remarkably, a small number of VAAVs appeared to encapsulate two AAVs at once (FIG. 5B). By comparison, AAV9-VP2-SpT3 which had been reacted with SpC3 (without the INT peptide) did not undergo packaging into vaults (FIG. 5C). These technical results encouraged us to further develop VAAV as a gene therapy delivery system.

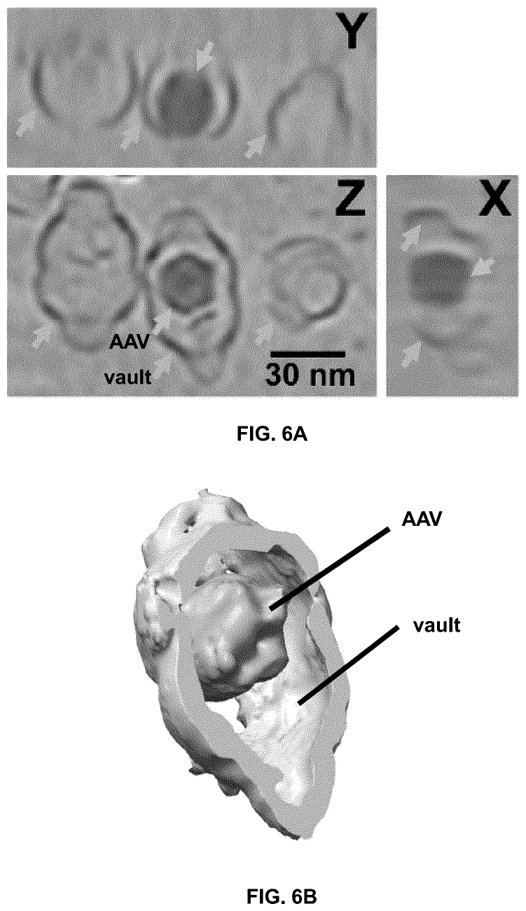

To locate AAVs more precisely within vaults, cryogenic electron tomography (cryoET) was performed on VAAV samples. Tomogram slices show the distribution of vaults and AAVs in three orthogonal directions (FIG. 6A). Three vaults with different orientations are recognizable in the slices. The hexagonal density (green arrows) with darker contrast comes from the icosahedral AAV, which resides exactly inside the central cavity of the vault from all three views. The 3D rendering of this VAAV particle (FIG. 6B) shows that the AAV is well encapsulated by the vault shell. Vault structures shown in both tomogram slices and 3D rendering are not fully closed due to the missing wedge reconstruction issue, which made the whole density distort and elongate in the Z direction. The cryoET data provided direct evidence that AAVs are indeed packaged inside vaults.

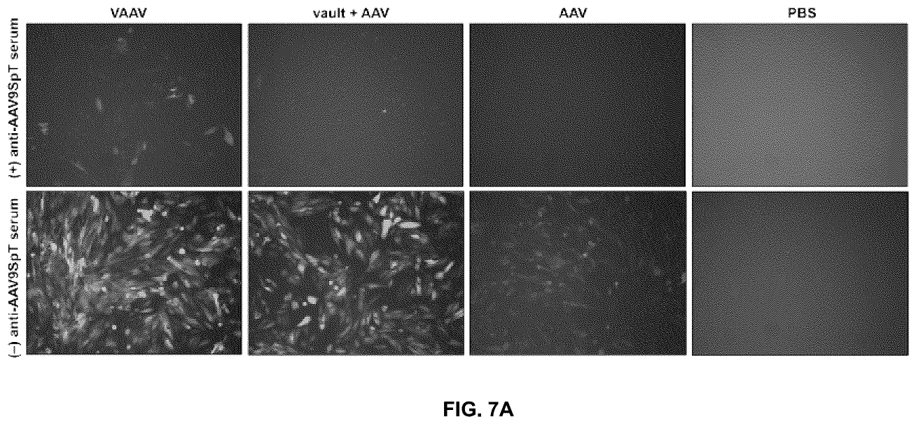

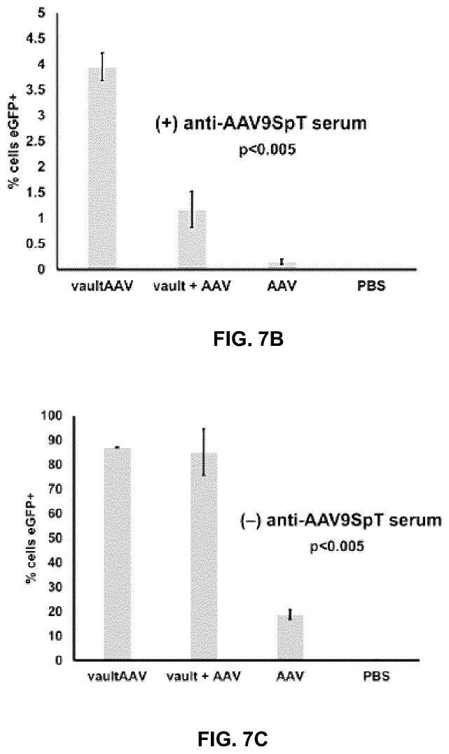

As a proof-of-principle to test the immunological shielding capacity of VAAV, we utilized neutralizing serum from mice immunized against AAV9-VP2-SpT3. We first performed a neutralization assay to verify that the serum completely blocked AAV9-VP2-SpT3 transduction of cultured CHO-Lec2 cells. Then we treated CHO-Lec2 cells with VAAV, vault+AAV, AAV9-INT alone, and PBS both in the presence and the absence of a fully neutralizing level of serum. Even with neutralizing serum, VAAV successfully transduced cells (FIG. 7A, FIG. 7B). Roughly speaking, the percentage of transduction paralleled the amount of packaged VAAVs we observed in the TEM images. By contrast, vault+AAV showed a very low level of transduction, AAV alone did not transduce cells, and no fluorescence was observed in the PBS group. In the absence of neutralizing serum, VAAV and vault+AAV very strongly transduced cells, AAV alone transduced cells to a moderate extent, and no fluorescence was observed in the PBS group (FIG. 7A, FIG. 7C). Enhanced vault+AAV transduction might arise from nonspecific associations between vault and AAV bringing AAV “along for the ride” when vault undergoes endocytic uptake into the cells. It is noteworthy that similar enhancement has been observed in the context of vaults improving transfection of nonpackaged plasmids and has previously been termed the “bystander effect”. These data demonstrate that VAAV protects its AAV cargo from neutralizing serum as well as enhances cellular transduction, allowing for the delivery of AAV gene therapy despite neutralizing antibodies.

### Discussion

AAV gene therapy has long been plagued by the neutralizing antibody problem and solutions have so far been limited. VAAV offers a promising route for attacking this problem. VAAV protects AAV capsids via the immunologically invisible vault shell, facilitating successful delivery even when neutralizing antibodies are present. We anticipate that this platform technology will possess wide applicability since even though vault has a natural tropism for phagocytic cells, one can retarget the complex by attaching peptides or antibodies. Our proof-of-principle data showcase the potential of VAAV for circumventing the neutralizing antibody problem.

In addition, we demonstrated that mixtures of vaults and AAVs exhibit greatly enhanced cellular transduction efficiency regardless of whether AAVs are packaged into vaults. This might arise from vaults nonspecifically associating with AAVs and thus taking them along during endocytosis or the previously mentioned “bystander effect”. Enhanced VAAV transduction might help alleviate the need for utilizing potentially toxic high doses of AAV. Lower doses of AAV also may ease the manufacturing burden and thus decrease the presently high costs of treatments. The strength of the effect in CHO-Lec2, a cell type known to endocytose vaults with a relatively modest degree of efficiency, supports the idea that the effect may not depend on cell type-specific mechanisms. Cell types that endocytose vaults more efficiently (e.g. phagocytes) may thus show the same or an even stronger transduction enhancing effect. It should be noted that this will likely still necessitate creating an AAV-SpT3 serotype which experiences efficient trafficking to the nucleus of the target cell type after endocytic uptake. VAAV's enhanced transduction is an exciting property that will benefit from further exploration.

Despite vault's tropism for phagocytic cells, past investigations have shown that it can be retargeted to transduce a variety of useful tissues. As an example, an epidermal growth factor (EGF) peptide was linked to the C-terminus of MVP within vaults, successfully targeting uptake into epithelial cancer cells. The fusion of an Fc-binding protein to MVP along with the addition of an anti-EGFR antibody enabled targeting of epithelial cancer cells. These modified vaults experienced minimal uptake into non-target cell types. As such, VAAV should be amenable to the same sorts of powerful targeting techniques. VAAV thus might gain footing as a highly versatile platform technology with the capacity for both immunological shielding and tissue-specific targeting, making it useful for a wide variety of biomedical applications.

VAAV offers a solution to the neutralizing antibody problem of AAV gene therapy and further improves cellular transduction efficiency (FIG. 1A, FIG. 1B, FIG. 1C, FIG. 1D). SpyTag-SpyCatcher molecular glue peptides are used as a method of site-specific conjugation for the INT peptide (FIG. 2C, FIG. 2D, FIG. 2E), which binds to the interior of the vault and facilitates packaging. VAAV complexes were imaged via TEM and showed a large fraction of AAVs packaged inside of vaults (FIG. 5A, FIG. 5B, FIG. 5C). The packaging of AAVs within the vault interior was further verified using cryoET (FIG. 6A, FIG. 6B). AAV9-INT represents the largest cargo and the first virus packaged inside of a vault. VAAV was shown to transduce cells in the presence of neutralizing serum and VAAV in general exhibited a transduction efficiency enhancing effect (FIG. 7A, FIG. 7B, FIG. 7C). VAAV could expand the scope of AAV medicines to treat patient populations who have preexisting neutralizing antibodies and might eventually enable redosable AAV therapeutics.

What is claimed is:

1. A composition for delivery of an AAV therapy, the composition comprising an adeno-associated virus (AAV) vector packaged inside a vault nanoparticle, wherein:
a. the AAV vector comprises a protein capsid functionalized with a plurality of INT proteins;
b. the vault nanoparticle comprises a plurality of MVP proteins; and
c. an INT moiety of the AAV vector is covalently attached near an N-terminus of an MVP protein.

2. The composition of claim 1, wherein the protein capsid comprises a plurality of SpyTag proteins, a plurality of SpyCatcher proteins covalently attached to the plurality of Spytag proteins, and a plurality of INT proteins covalently attached to the plurality of SpyCatcher proteins.

3. The composition of claim 2, wherein the SpyTag protein comprises SpyTag003 protein and the SpyCatcher protein comprises SpyCatcher003 protein.

4. The composition of claim 3, wherein the SpyTag003 protein is flanked by a first and second flexible linker comprising the amino acid sequences AGGGSGGS and AGGGSGGS, respectively.

5. The composition of claim 3, wherein the INT peptide is covalently attached to the SpyCatcher003 with a third flexible linker.

6. The composition of claim 1, wherein the AAV vector further comprises a vector genome comprising a therapeutic gene.

7. The composition of claim 6, wherein the therapeutic gene is selected from a gene therapy for a genetic disease or disorder and a gene encoding a vaccine antigen.

8. The composition of claim 1, wherein the vault nanoparticle is further functionalized with a plurality of targeting molecules configured to preferentially bind to corresponding target cells.

9. A method of treating a patient in need of an AAV therapy, the method administering a therapeutically effective amount of a composition comprising an adeno-associated virus (AAV) vector packaged inside a vault nanoparticle, wherein:
a. the AAV vector comprises a protein capsid functionalized with a plurality of INT proteins;
b. the vault nanoparticle comprises a plurality of MVP proteins; and
c. an INT moiety of the AAV vector is covalently attached near an N-terminus of an MVP protein.

10. The method of claim 9, wherein the protein capsid comprises a plurality of SpyTag proteins, a plurality of SpyCatcher proteins covalently attached to the plurality of Spytag proteins, and a plurality of INT proteins covalently attached to the plurality of SpyCatcher proteins.

11. The method of claim 10, wherein the SpyTag protein comprises SpyTag003 protein and the SpyCatcher protein comprises SpyCatcher003 protein.

12. The method of claim 11, wherein the SpyTag003 protein is flanked by a first and second flexible linker comprising the amino acid sequences AGGGSGGS and AGGGSGGS, respectively.

13. The method of claim 11, wherein the INT peptide is covalently attached to the SpyCatcher003 with a third flexible linker.

14. The method of claim 9, wherein the AAV vector further comprises a vector genome comprises a therapeutic gene.

15. The composition of claim 6, wherein the therapeutic gene is selected from a gene therapy for a genetic disease and a gene encoding a vaccine antigen.

16. The method of claim 9, wherein the vault nanoparticle is configured to prevent the AAV vector from a neutralizing antibody produced by an immune response to the AAV therapy.

17. The method of claim 9, wherein the vault nanoparticle is further functionalized with a plurality of targeting molecules configured to preferentially bind to corresponding target cells.

18. A method for producing an AAV therapy composition comprising an adeno-associated virus (AAV) vector packaged inside a vault nanoparticle, the method comprising:
a. providing a plurality of AAV vectors, each AAV vector comprising a protein capsid functionalized with a plurality of SpyTag proteins flanked by first and second flexible linkers;
b. providing a plurality of fusion proteins, each fusion protein comprising an INT protein covalently linked to a SpyCatcher protein by a third flexible linker;
c. providing a plurality of vault nanoparticles comprising a plurality of MVT proteins;
d. mixing the plurality of AAV vectors with the plurality of fusion proteins to form a plurality of INT-functionalized AAV vectors, each INT-functionalized AAV vector comprising an AAV vector and the SpyCatcher proteins of at least a portion of the fusion proteins covalently attached the SpyTag proteins of the protein capsid; and
e. incubating the INT-functionalized AAV vectors with the plurality of vault nanoparticles to form the AAV therapy composition, wherein at least a portion of the INT proteins of each INT-functionalized AAV vector is covalently attached to at least a portion of the MVP proteins of each vault nanoparticle.

19. The method of claim 18, wherein:
a. the plurality of AAV vectors are mixed with the plurality of fusion proteins at a copy ratio of 1 copy of an AAV vector to 10 copies of the fusion protein; and
b. the INT-functionalized AAV vectors are incubated with the vault nanoparticles at a copy ratio of 1 copy of the INT-functionalized AAV vector to 5 copies of the vault nanoparticles.

20. The method of claim 18, wherein the SpyTag protein comprises SpyTag003 protein, the SpyCatcher protein comprises SpyCatcher003 protein, and the first and second flexible linkers comprise the amino acid sequences AGGGSGGS and AGGGSGGS, respectively.
